(19) United States
(12) Patent Application Publication
(10) Pub. No.: US 2008/0306073 A1
(43) Pub. Date: Dec. 11, 2008

(54) Antitumor Effect Potentiator, Antitumor Preparation, and Method for Treating Cancer

(76) Inventors: Katsuhisa Koizumi, Tokushima (JP); Junji Uchida, Tokushima (JP); Teiji Takechi, Tokushima (JP); Mamoru Nukatsuka, Tokushima (JP)

Correspondence Address: ROTHWELL, FIGG, ERNST & MANBECK, P.C., 1425 K STREET, N.W., SUITE 800, WASHINGTON, DC, 20005, US

(21) Appl. No.: 11/629,185

(22) PCT Filed: Jun. 2, 2005
(86) PCT No.: PCT/JP05/10182

§ 371 Date: Mar. 9, 2007

(51) Int. Cl. A61K 31/53; C07F 15/00; A61P 35/00

(52) U.S. Cl. 514/241; 556/136

(57) ABSTRACT

An antitumor effect fortifier comprising as an active ingredient an effective amount of platinum (II) cis-oxalato(1R,2R-diaminocyclohexane) so as to enhance the antitumor activity of antitumor agent containing a therapeutically effective amount of tegafur, an amount, effective for the enhancement of antitumor effect, of gimeracil and an amount, effective for the suppression of side effects, of oteracil potassium; and a relevant antitumor agent. Further, there is provided a method of therapy for cancer, characterized in that an amount, effective for the enhancement of antitumor effect, of platinum (II) cis-oxalato(1R,2R-diaminocyclohexane) is concomitantly administered to a mammal. Still further, there is provided an antitumor agent consisting of a pharmaceutical preparation form composed of at least two separate preparations containing efficacious ingredients consisting of tegafur, gimeracil, oteracil potassium and platinum (II) cis-oxalato(1R,2R-diaminocyclohexane) each alone or in an arbitrary combination, or a pharmaceutical preparation form constituted of a single preparation containing all the efficacious ingredients. Still further, there is provided a relevant kit.

### TECHNICAL FIELD

The present invention relates to an antitumor effect potentiator; a method for treating cancer by potentiating an antitumor effect according to a novel concomitant administration of an antitumor preparation; an antitumor preparation; and a kit for treating cancer.

### BACKGROUND ART

The research and development of antitumor preparations has been actively carried out. A variety of effective antitumor preparations are clinically used in the treatment of malignant tumors. For example, tegafur is a drug that is activated in vivo and gradually releases the active principle, i.e., 5-fluorouracil (hereinafter referred to as “5-FU”), thereby lessening the toxicity and side effects presented by 5-FU.

A compound pharmaceutical agent of this tegafur and uracil (trade name: UFT, molar ratio of tegafur/uracil=1:4, manufactured by Taiho Pharmaceutical Co., Ltd.) is known. This compound pharmaceutical agent exhibits a significant antitumor effect due to the fact that uracil, which does not have any antitumor effect by itself, inhibits the inactivation of 5-FU which when used alone is promptly metabolized and inactivated in vivo.

A 3-membered compound pharmaceutical agent containing tegafur, gimeracil, and oteracil potassium (trade name: TS-1, molar ratio of tegafur/gimeracil/oteracil potassium=1:0.4:1, manufactured by Taiho Pharmaceutical Co., Ltd.) is also known. This compound pharmaceutical agent has a stronger antitumor effect due to the fact that gimeracil exhibits a 5-FU decomposition inhibitory action about 200 times greater than that of uracil. With respect to this compound pharmaceutical agent, oteracil potassium specifically inhibits the increase in gastrointestinal toxicity that is likely to be accompanied by the potentiation in antitumor effect attained by the two ingredients, i.e., tegafur and gimeracil, thereby potentiating the therapeutic effect. UFT and TS-1 therefore contribute to the treatment of various malignant tumors.

Drugs and therapeutic methods that give a stronger therapeutic effect are still required such that the survival of cancer patients is further prolonged. One example of a therapeutic method that has long been used to achieve such an object is administering in combination a plurality of drugs that have different mechanisms of expressing antitumor effects and different side effects so as to improve the therapeutic result (a combination therapy). Some combination therapies contribute to improving therapeutic results (see, for example, patent publications 1, 2, 3, and 4). For example, oxaliplatin when used alone exhibits a low antitumor effect, and is therefore used in combination therapies with other pharmaceutical agents. Combination therapies using 5-fluorouracil and calcium folinate (FOLFOX) are commonly used worldwide (see, for example, non-patent publications 1, 2, and 3). FOLFOX requires complicated procedures and are thus problematic in being detrimental to the QOL of patients due to the physical restraint accompanied by continuous infusion, high medical costs, etc. Therefore, development of better combination therapies using oxaliplatin has been attempted all over the world. As an example, a combination therapy using oxaliplatin and capecitabine (XELOX), which is a fluorinated pyrimidine-based anticancer agent (trade name: Xeroda), is reported to give an antitumor effect almost identical to FOLFOX (see, for example, non-patent publication 4). However, combination therapies that give a stronger therapeutic effect are still required.
Patent Publication 1: Japanese Patent Publication No. 2557303
Patent Publication 2: Japanese Patent Publication No. 2614164
Patent Publication 3: Japanese Unexamined Patent Publication No. 1996-169825
Patent Publication 4: Japanese Unexamined Patent Publication No. 2002-205945
Non-Patent Publication 1: Journal of Clinical Oncology, Vol. 22, 22-30, 2004
Non-Patent Publication 2: Journal of Clinical Oncology, Vol. 21, 2059-2069, 2004
Non-Patent Publication 3: Journal of Clinical Oncology, Vol. 18, 2938-2947, 2000
Non-Patent Publication 4: Journal of Clinical Oncology, Vol. 22, 2084-2091, 2004

### DISCLOSURE OF THE INVENTION

A primary object of the invention is to provide an antitumor effect potentiator for a tegafur/gimeracil/oteracil potassium compound pharmaceutical agent; a method for treating cancer that gives an excellent therapeutic effect due to the use of a specific pharmaceutical agent in combination with the compound pharmaceutical agent; an antitumor preparation containing the specific pharmaceutical agent and the compound pharmaceutical agent; and a kit.

In view of the current state of the art described above, the inventors studied a novel combination therapy with a 3-membered compound pharmaceutical agent containing tegafur, gimeracil, and oteracil potassium in combination with another antitumor preparation to develop a method for treating cancer that strongly contributes to prolonging the survival of patients, and as a result, found that the use of a platinous complex, i.e., cis-oxalate(1R,2R-diaminocyclohexane)platinum(II) (generic name: oxaliplatin, trade names: Eloxatin and Elplat, hereinafter referred to as l-OHP) in combination with the aforementioned 3-membered compound pharmaceutical agent significantly potentiates the antitumor effect without aggravating side effects. The inventors confirmed that the tumor growth inhibitory effect attained by this novel combination therapy is superior to that of standard chemotherapies for cancer of the large intestine such as a combination therapy using a tegafur/uracil compound pharmaceutical agent and a salt of d,l-folinic acid (see Japanese Patent Publication No. 2557303) and a combination therapy using a tegafur/uracil compound pharmaceutical agent, a salt of d,l-folinic acid, and l-OHP (see U.S. Pat. No. 6,602,870). The present invention has been accomplished based on these novel findings.

In particular, the present invention provides antitumor effect potentiators, methods for treating cancer, antitumor preparations, and an antitumor preparation-containing kit, methods for potentiating an antitumor effect, etc., as described below.

1. An antitumor effect potentiator for potentiating antitumor activity of an antitumor preparation comprising tegafur in a therapeutically effective amount, gimeracil in an amount effective for potentiating an antitumor effect, and oteracil potassium in an amount effective for inhibiting a side effect,

the antitumor effect potentiator comprising cis-oxalate(1R,2R-diaminocyclohexane)platinum(II) in an amount effective for potentiating an antitumor effect.

2. The antitumor effect potentiator according to Item 1, wherein the antitumor preparation comprises tegafur, gimeracil, and oteracil potassium in a molar ratio of 1:0.4:1.

3. A method for treating cancer comprising the step of administering to a mammal the combination of tegafur in a therapeutically effective amount, gimeracil in an amount effective for potentiating an antitumor effect, oteracil potassium in an amount effective for inhibiting a side effect, and cis-oxalate(1R,2R-diaminocyclohexane)platinum(II) in an amount effective for potentiating an antitumor effect.

4. The method for treating cancer according to Item 3, wherein, per mole of tegafur, gimeracil is used in a proportion of 0.1 to 5 mole, oteracil potassium is used in a proportion of 0.1 to 5 mole, and cis-oxalate(1R,2R-diaminocyclohexane)platinum(II) is used in a proportion of 0.1 to 5 mole.

5. The method for treating cancer according to Item 4, wherein tegafur, gimeracil, oteracil potassium, and cis-oxalate(1R,2R-diaminocyclohexane)platinum(II) are used in a molar ratio of 1:0.4:1:0.1 to 5.

6. An antitumor preparation in a pharmaceutical form comprising a plurality of pharmaceutical agents each of which contains one active ingredient selected from the group consisting of tegafur, gimeracil, oteracil potassium, and cis-oxalate(1R,2R-diaminocyclohexane)platinum(II), or each of which contains such active ingredients in any combination, or in a pharmaceutical form comprising a single pharmaceutical agent containing all of the active ingredients.

7. The antitumor preparation according to Item 6 in a pharmaceutical form comprising a compound pharmaceutical agent comprising 3 ingredients, i.e., tegafur, gimeracil, and oteracil potassium, as active ingredients, and a pharmaceutical agent comprising cis-oxalate(1R,2R-diaminocyclohexane)platinum(II) as an active ingredient.

8. The antitumor preparation according to Item 6 or 7, wherein the proportion of the active ingredients is such that, per mole of tegafur, gimeracil is used in a proportion of 0.1 to 5 mole, oteracil potassium is used in a proportion of 0.1 to 5 mole, and cis-oxalate(1R,2R-diaminocyclohexane)platinum(II) is used in a proportion of 0.1 to 5 mole.

9. The antitumor preparation according to Item 8, wherein the molar ratios of the active ingredients are such that tegafur, gimeracil, oteracil potassium, and cis-oxalate(1R,2R-diaminocyclohexane)platinum(II) are used in a molar ratio of 1:0.4:1:0.1 to 5.

10. A kit comprising a combination of pharmaceutical compositions for treating cancer in a mammal comprising (a) an antitumor composition comprising tegafur in a therapeutically effective amount, gimeracil in an amount effective for potentiating an antitumor effect, and oteracil potassium in an amount effective for inhibiting a side effect, and (b) cis-oxalate(1R,2R-diaminocyclohexane)platinum(II) in an amount effective for potentiating an antitumor effect.

11. A method for potentiating an antitumor effect of an antitumor preparation, the method comprising the step of administering cis-oxalate(1R,2R-diaminocyclohexane)platinum(II) in an amount effective for potentiating an antitumor effect in combination with an antitumor preparation comprising tegafur in a therapeutically effective amount, gimeracil in an amount effective for potentiating an antitumor effect, and oteracil potassium in an amount effective for inhibiting a side effect.

12. The method according to Item 11, wherein cis-oxalate(1R,2R-diaminocyclohexane)platinum(II) is administered concurrently with, or within 4 hours before or after, the administration of the antitumor preparation comprising tegafur in a therapeutically effective amount, gimeracil in an amount effective for potentiating an antitumor effect, and oteracil potassium in an amount effective for inhibiting a side effect.

13. Use of cis-oxalate(1R,2R-diaminocyclohexane)platinum(II) in production of an antitumor effect potentiator to potentiate an antitumor effect of an antitumor preparation comprising tegafur in a therapeutically effective amount, gimeracil in an amount effective for potentiating an antitumor effect, and oteracil potassium in an amount effective for inhibiting a side effect.

14. Use of cis-oxalate(1R,2R-diaminocyclohexane)platinum(II) in production of an antitumor preparation comprising tegafur in a therapeutically effective amount, gimeracil in an amount effective for potentiating an antitumor effect, and oteracil potassium in an amount effective for inhibiting a side effect, to produce an antitumor preparation that has an potentiated antitumor effect.

The antitumor effect potentiator of the present invention contains cis-oxalate (1R,2R-diaminocyclohexane)platinum(II) as an active ingredient. Due to the antitumor effect potentiator, the antitumor effect of an antitumor preparation containing 3 ingredients, i.e., tegafur, gimeracil, and oteracil potassium, as active ingredients can be significantly potentiated.

A feature of the method for treating cancer of the present invention is concomitantly administering to a mammal tegafur in a therapeutically effective amount, gimeracil in an amount effective for potentiating an antitumor effect, oteracil potassium in an amount effective for inhibiting a side effect, and cis-oxalate(1R,2R-diaminocyclohexane)platinum(II) in an amount effective for potentiating an antitumor effect.

A feature of the antitumor preparation of the present invention is being in a pharmaceutical form comprising a plurality of pharmaceutical agents each of which contains one active ingredient selected from the group consisting of tegafur, gimeracil, oteracil potassium, and cis-oxalate(1R,2R-diaminocyclohexane)platinum(II), or each of which contains such active ingredients in any combination, or in a pharmaceutical form comprising a single pharmaceutical agent containing all these active ingredients.

A feature of the kit of the present invention is comprising a combination of pharmaceutical compositions for treating cancer in a mammal comprised of (a) an antitumor composition comprising tegafur in a therapeutically effective amount, gimeracil in an amount effective for potentiating an antitumor effect, and oteracil potassium in an amount effective for inhibiting a side effect, and (b) cis-oxalate(1R,2R-diaminocyclohexane)platinum(II) in an amount effective for potentiating an antitumor effect.

A feature of the method for potentiating an antitumor effect of the present invention is administering cis-oxalate(1R,2R-diaminocyclohexane)platinum(II) in an amount effective for potentiating an antitumor effect in combination with an antitumor preparation comprising tegafur in a therapeutically effective amount, gimeracil in an amount effective for potentiating an antitumor effect, and oteracil potassium in an amount effective for inhibiting a side effect.

l-OHP used as an active ingredient of the antitumor effect potentiator is a platinum-containing complex, and is a known compound. l-OHP induces functional disorder of DNA and DNA strand breakage by binding to the DNA of cancer cells, thereby exerting an action of annihilating cancer cells. l-OHP can be produced according to known methods, for example, the method disclosed in Japanese Examined Patent Publication No. 1985-41077.

Tegafur (generic name, chemical name: 5-fluoro-1-(2-tetrahydrofuryl)-2,4-(1H,3H)-pyrimidinedione, hereinafter sometimes referred to as FT), an active ingredient of the antitumor preparation, is a known compound, and it is a drug activated in vivo and releases an active principle, i.e., 5-FU, thereby revealing an antitumor activity. Tegafur can be produced according to known methods, for example, the method disclosed in Japanese Examined Patent Publication No. 1974-10510.

Gimeracil (generic name, chemical name: 2,4-dihydroxy-5-chloropyridine, hereinafter sometimes referred to as CDHP) is also a known compound, and although it does not exhibit any antitumor activity by itself, it can potentiate an antitumor effect by inhibiting the in vivo metabolic inactivation of 5-FU.

Oteracil potassium (generic name, chemical name: monopotassium 1,2,3,4-tetrahydro-2,4-dioxo-1,3,5-triazine-6-carboxylate, hereinafter sometimes referred to as OXO) is also a known compound. Although it does not exhibit any antitumor activity by itself, it mostly remains in the gastrointestinal tract and inhibits the activation of 5-FU at that location, thereby preventing gastrointestinal tract disorders caused by 5-FU.

With respect to the antitumor preparation containing 3 ingredients, i.e., tegafur, gimeracil, and oteracil potassium, as active ingredients, the proportion of each active ingredient may be within the ranges described in connection with a known compound pharmaceutical agent, for example, that disclosed in Patent Publication No. 2614164. It is usually such that, per mole of tegafur, gimeracil is used in a proportion of about 0.1 to about 5 mole and preferably about 0.2 to about 1.5 mole, and oteracil potassium is used in a proportion of about 0.1 to about 5 mole and preferably about 0.2 to about 2 mole. A particularly preferable proportion of the 3 ingredients is tegafur:gimeracil:oteracil potassium=1:0.4:1 in molar ratio (hereinafter, a compound pharmaceutical agent containing the ingredients in this ratio is sometimes referred to as TS-1).

The antitumor preparation containing tegafur, gimeracil, and oteracil potassium as active ingredients may be prepared in a pharmaceutical form comprising two or more pharmaceutical agents each of which contains one of the active ingredients, or each of which contains such active ingredients in any combination, or in a pharmaceutical form comprising a single pharmaceutical agent containing all of the active ingredients. In either case, such antitumor preparations are prepared as pharmaceutical compositions according to standard methods using suitable pharmaceutical carriers. Carriers usable herein are those that are commonly used in conventional drugs, for example, excipients, binders, disintegrators, lubricants, colorants, taste enhancers, flavor enhancers, surfactants, etc.

When an antitumor preparation in a pharmaceutical form comprising two or more pharmaceutical agents as described above is used, each pharmaceutical agent may be administered concurrently, or one pharmaceutical agent may be administered any time before or after the administration of the other pharmaceutical agent(s). Preferably, all of the pharmaceutical agents are administered concurrently, or one pharmaceutical agent is administered within 4 hours, and more preferably within 2 hours, before or after the administration of the other pharmaceutical agent(s).

The antitumor effect potentiator comprising l-OHP as an active ingredient of the present invention may be prepared singly in a unit dosage form. In this case, the antitumor effect potentiator is prepared as a pharmaceutical composition according to standard methods using suitable pharmaceutical carriers. Carriers usable herein are those that are commonly used in conventional drugs, for example, excipients, binders, disintegrators, lubricants, colorants, taste enhancers, flavor enhancers, surfactants, etc. The antitumor effect potentiator prepared in any unit dosage form may be administered concurrently with, or before or after, the antitumor preparation containing 3 ingredients, i.e., tegafur, gimeracil, and oteracil potassium, as active ingredients that may also be prepared in any unit dosage form. That is, the antitumor effect potentiator of the present invention can be administered any time before or after or concurrently with the administration of the antitumor preparation containing 3 ingredients, i.e., tegafur, gimeracil, and oteracil potassium, as active ingredients. Preferably, the antitumor effect potentiator is administered concurrently with or within 4 hours before or after the administration of the antitumor preparation, and preferably within 2 hours, before or after the administration of the antitumor preparation.

When the antitumor effect potentiator of the present invention is administered concurrently with, or before or after, the aforementioned antitumor preparation containing 3 ingredients, i.e., tegafur, gimeracil, and oteracil potassium, as active ingredients, the antitumor effect potentiator is preferably administered in an amount such that the amount of l-OHP, per mole of tegafur, is within the range of about 0.1 to about 5 mole, preferably about 0.3 to about 3 mole, and more preferably about 0.4 to about 1 mole.

Unit dosage forms usable for administering the antitumor effect potentiator of the present invention to treat malignant tumors of mammals, including humans, are not limited, and can be suitably selected according to the purpose of the treatment. Specific examples are injections, suppositories, ophthalmic solutions, ointments, aerosols, and like parenteral forms; tablets, coated tablets, powders, granules, capsules, fluids, pills, suspensions, emulsions, and like oral forms, with injections being of a preferable form of administration. The antitumor effect potentiator can be produced in such dosage forms according to methods commonly known in this technical field.

According to the present invention, an antitumor preparation containing an antitumor effect potentiator can be prepared, in which l-OHP, which is the active ingredient of the aforementioned antitumor effect potentiator, is concomitantly present with an antitumor preparation containing 3 ingredients, i.e., tegafur, gimeracil, and oteracil potassium, as active ingredients. Such an antitumor preparation may be in a pharmaceutical form comprising a plurality of pharmaceutical agents each of which contains one of the aforementioned 4 ingredients, or each of which contains such ingredients in any combination, or in a pharmaceutical form comprising a single pharmaceutical agent containing all of the ingredients. In other words, such an antitumor preparation may be a drug comprising a single portion consisting of a pharmaceutical agent containing all of the aforementioned 4 ingredients, or may be a drug comprising multiple portions consisting of a pharmaceutical agent containing 1 to 3 ingredients and a pharmaceutical agent containing another ingredient. In particular, a 2-part preparation in which a compound pharmaceutical agent containing 3 ingredients, i.e., tegafur, gimeracil, and oteracil potassium, as active ingredients and a pharmaceutical agent containing l-OHP as an active ingredient are present as separate portions is preferable.

With respect to the antitumor preparation, the proportion of the ingredients is not limited, irrespective of being composed of a single pharmaceutical agent or multiple pharmaceutical agents. Usually, per mole of tegafur, gimeracil is used in a proportion of about 0.1 to about 5 mole and preferably about 0.2 to about 1.5 mole, oteracil potassium is used in a proportion of about 0.1 to about 5 mole and preferably about 0.2 to about 2 mole, and l-OHP is used in a proportion of about 0.1 to about 5 mole, preferably about 0.3 to about 3 mole and more preferably about 0.4 to about 1 mole. In particular, a preferable molar ratio of the ingredients is tegafur:gimeracil:oteracil potassium:l-OHP=about 1:0.4:10.1 to 5, more preferably about 1:0.4:1:0.3 to 3, and particularly preferably about 1:0.4:1:0.4 to 1. When the antitumor preparation is a 2-part drug in which a compound pharmaceutical agent containing 3 ingredients, i.e., tegafur, gimeracil, and oteracil potassium, as active ingredients and a pharmaceutical agent containing l-OHP as an active ingredient are present as separate portions as described above, the antitumor preparation preferably contains the compound pharmaceutical agent containing tegafur, gimeracil, and oteracil potassium in a molar ratio of 1:0.4:1, and the pharmaceutical preparation containing, per mole of tegafur, l-OHP in a proportion of about 0.1 to about 5 mole, preferably about 0.3 to about 3 mole, and more preferably about 0.4 to about 1 mole.

The active ingredients may be prepared as pharmaceutical compositions according to standard methods using suitable pharmaceutical carriers. Carriers usable herein are those that are commonly used in conventional drugs, for example, excipients, binders, disintegrators, lubricants, colorants, taste enhancers, flavor enhancers, surfactants, etc.

When a multi-part antitumor preparation comprising two or more pharmaceutical agents as described above is used, each pharmaceutical agent may be administered concurrently, or each pharmaceutical agent may be administered any time before or after the administration of the other pharmaceutical agent(s). Preferably, all of the pharmaceutical agents are administered concurrently, or one pharmaceutical agent is administered within 4 hours, and more preferably within 2 hours, before or after the administration of the other pharmaceutical agents.

According to the present invention, the l-OHP-containing pharmaceutical agent and the compound pharmaceutical agent containing tegafur, gimeracil, and oteracil potassium described above are applicable to a kit comprising a combination of pharmaceutical compositions for mammalian cancer treatment comprising:
(a) an antitumor composition containing tegafur in a therapeutically effective amount, gimeracil in an amount effective for potentiating an antitumor effect, and oteracil potassium in an amount effective for inhibiting a side effect, and
(b) a composition containing l-OHP in an amount effective for potentiating an antitumor effect. The compositions contained in such a kit may be in any known pharmaceutical form. The compositions are usually housed in any commonly-used containers according to their pharmaceutical form.

Such a kit is applicable to, for example, a kit for mammalian cancer treatment containing at least 4 ingredients consisting of:
(i) tegafur in a therapeutically effective amount,
(ii) gimeracil in an amount effective for potentiating an antitumor effect,
(iii) oteracil potassium in an amount effective for inhibiting a side effect, these all being part of an antitumor composition, and
(iv) l-OHP in an amount effective for potentiating an antitumor effect, and
   at least 2 containers for housing these ingredients, in which tegafur and l-OHP are housed in separate containers. The aforementioned ingredients (i) to (iv) are preferably in a pharmaceutical form prepared in combination with pharmaceutically acceptable carriers. With respect to the aforementioned kit, insofar as ingredients (i) and (iv) are housed in separate containers, ingredients (ii) and (iii) may be independently housed in containers that are separate from those in which the aforementioned two ingredients are housed, or ingredients (ii) and (iii) may be independently mixed with ingredient (i) or (iv) for housing in the same container. A preferable kit is such that a pharmaceutical agent containing ingredients (i) to (iii) is housed in one container and a pharmaceutical agent containing ingredient (iv) is housed in another container.

Unit dosage forms usable in administering the antitumor preparation of the present invention to treat malignant tumors of mammals, including humans suffering malignant tumors, are not limited, and can be selected according to the purpose of the treatment. Specific examples are injections, suppositories, ophthalmic solutions, ointments, aerosols, and like parenteral forms; and tablets, coated tablets, powders, granules, capsules, fluids, pills, suspensions, emulsions, and like oral forms. The antitumor preparation can be produced in such dosage forms according to methods commonly known in this technical field.

In connection with the antitumor effect potentiator and the antitumor preparation of the present invention, in preparing solid oral agents, such as tablets, powders, and granules, for example, the following can be used as carriers: lactose, saccharose, sodium chloride, glucose, urea, starch, calcium carbonate, kaolin, crystalline cellulose, silicic acid, methylcellulose, glycerol, sodium alginate, gum arabic, and like excipients; simple syrups, liquid glucose, liquid starch, gelatin solutions, polyvinyl alcohol, polyvinyl ether, polyvinylpyrrolidone, carboxymethylcellulose, shellac, methylcellulose, ethylcellulose, water, ethanol, potassium phosphate, and like binders; dried starch, sodium alginate, powdered agar, powdered laminaran, sodium hydrogencarbonate, calcium carbonate, polyoxyethylene sorbitan fatty acid esters, sodium lauryl sulfate, stearic acid monoglycerides, starch, lactose, and like disintegrators; saccharose, stearic acid, cocoa butter, hydrogenated oils, and like disintegration inhibitors; sodium lauryl sulfate, and like absorption enhancers; glycerol, starch, and like humectants; starch, lactose, kaolin, bentonite, colloidal silicic acid, and like adsorbents; purified talc, stearic acid salts, powdered boric acid, polyethylene glycol, and like lubricants; etc. Furthermore, tablets may be provided with a standard coating as necessary, such as sugar-coated tablets, gelatin-coated tablets, enteric-coated tablets, film-coated tablets, double-layer tablets, multi-layer tablets, and the like.

In preparing pills, glucose, lactose, starch, cacao butter, hydrogenated vegetable oils, kaolin, talc, and like excipients; powdered gum arabic, powdered tragacanth, gelatin, and like binders; laminaran, agar, and like disintegrants; etc., can be used as carriers.

Capsules can be prepared by mixing the active ingredients with the aforementioned various carriers and filling hard gelatin capsules, soft capsules, or the like, with the mixture.

In preparing suppositories, polyethylene glycol, cacao butter, lanolin, higher alcohols, esters of higher alcohols, gelatin, semisynthetic glycerides, Witepsol (registered trademark, Dynamite Nobel Inc.), etc., can be used as carriers.

In preparing injections, water, ethyl alcohol, macrogol, propylene glycol, ethoxylated isostearyl alcohol, polyoxylated isostearyl alcohol, polyoxyethylene sorbitan fatty acid esters, and like diluents; sodium citrate, sodium acetate, sodium phosphate, and like pH-adjusters; dipotassium phosphate, trisodium phosphate, sodium hydrogen phosphate, sodium citrate, and like buffers; sodium pyrosulfite, EDTA, thioglycolic acid, thiolactic acid, and like stabilizers; and mannitol, inositol, maltose, sucrose, lactose, and like saccharides for binders in freeze-drying; etc., can be used as carriers. In such cases, glucose and glycerol may be used in the pharmaceutical preparation in amounts sufficient to prepare an isotonic solution. Moreover, standard auxiliary dissolvents, soothing agents, topical anesthetics, etc., may be used. Subcutaneous, intramuscular, and intravenous injections can be prepared according to standard methods in conjunction with such carriers.

Liquid preparations may take a form of water-based or oil-based suspensions, solutions, syrups, or elixirs, and can be prepared according to standard methods using commonly-used additives.

In preparing the pharmaceutical preparation in a form of ointments such as pastes, creams, and gels, for example, white petrolatum, paraffin, glycerol, cellulose derivatives, polyethylene glycol, silicon, bentonite, etc., can be used as diluents.

The amounts of tegafur, gimeracil, oteracil potassium, and l-OHP, which are active ingredients of the antitumor preparation of the present invention, vary according to the dosage form, route of administration, dosing schedule, etc., and are not limited, and hence can be suitably selected. It is usually preferable that the proportion of the active ingredients accounts for about 1 to about 70 wt. % of the pharmaceutical preparation.

Methods for administration of the pharmaceutical preparation of the present invention are not limited and can be determined according to the form thereof, the age, sex, and condition of the patient, and other factors, and hence it may be administered enterally, orally, rectally, intraorally, intraarterially, intravenously, transdermally, or in a like manner. For example, tablets, pills, solutions, suspensions, emulsions, granules, capsules, and the like are administered orally; injections are administered intraarterially or intravenously; suppositories are administered intrarectally; and ointments are applied to the skin, the mucous membrane in the mouth, etc. With respect to the pharmaceutical preparation of the present invention, it is possible that the compound pharmaceutical agent containing tegafur, gimeracil, and oteracil potassium is orally administered while the l-OHP containing pharmaceutical agent is intravenously administered.

The dosage of each active ingredient in the present invention can be suitably selected according to the application, the age and sex of the patient, the degree of the disease, and other factors. The antitumor effect potentiator and the antitumor preparation of the present invention can be administered in 1 to 4 doses per day.

In oral administration, the pharmaceutical preparation of the present invention is preferably given in an amount using the following ranges as a standard: the amount of tegafur is about 0.1 to about 100 mg/kg/day, preferably about 0.2 to about 40 mg/kg/day, and more preferably about 0.5 to about 20 mg/kg/day; the amount of gimeracil is about 0.02 to about 30 mg/kg/day, preferably about 0.05 to about 12 mg/kg/day, and more preferably about 0.1 to about 6 mg/kg/day; the amount of oteracil potassium is about 0.1 to about 100 mg/kg/day, preferably about 0.2 to about 40 mg/kg/day, and more preferably about 0.5 to about 20 mg/kg/day; and the amount of l-OHP is about 0.08 to about 200 mg/kg/day, preferably about 0.15 to about 80 mg/kg/day, and more preferably about 0.4 to about 40 mg/kg/day.

When in the form of an injection, the pharmaceutical preparation, which may be diluted with aqueous glucose solution if necessary, can be gradually administered to an adult over 5 minutes or longer, usually in an amount corresponding to about 0.1 to about 100 mg/kg/day of tegafur and about 0.08 to about 200 mg/kg/day of l-OHP.

When in the form of a suppository, the pharmaceutical preparation of the invention is administered once or twice a day at an interval of 6 to 12 hours usually in an amount in an adult corresponding to about 0.1 to about 100 mg/kg/day of tegafur and about 0.08 to about 200 mg/kg/day of l-OHP by inserting it into the rectum.

The types of malignant tumors treatable by the administration of the pharmaceutical preparation of the present invention is not limited, insofar as the active principle, i.e., 5-FU, is reactive thereto; for example, head and neck cancer, stomach cancer, colon cancer, rectal cancer, liver cancer, gallbladder/biliary cancer, pancreatic cancer, lung cancer, breast cancer, vesical cancer, prostatic cancer, uterine cancer, esophageal cancer, renal cancer, ovarian cancer, etc. In particular, a remarkable effect can be expected from the pharmaceutical preparation of the present invention toward colon cancer, rectal cancer, breast cancer, esophageal cancer, stomach cancer, and head and neck cancer. Furthermore, a remarkable effect can be expected toward typical drug-resistant tumors and tumors that are starting to be drug-resistant.

Due to the antitumor effect potentiator, the method for treating cancer, the antitumor preparation, the kit, etc., of the present invention, an antitumor effect can be obtained that exceeds the effect obtained by a single use of a 3-membered compound pharmaceutical agent containing known antitumor drugs, e.g., tegafur, gimeracil, and oteracil potassium, and the effect obtained by a pharmaceutical agent containing l-OHP alone, without aggravating toxicity (gastrointestinal toxicity and bone marrow toxicity, in particular). Moreover, this antitumor effect is superior to the antitumor effect obtained by a combination therapy using a tegafur/uracil compound pharmaceutical agent and a salt of d,l-folinic acid, which is a standard therapy for cancer of the large intestine, and that obtained by a combination therapy using a tegafur/uracil compound pharmaceutical agent, a salt of d,l-folinic acid, and l-OHP. Furthermore, a remarkable antitumor effect potentiating action and antitumor effect can be expected from the pharmaceutical preparation of the present invention toward tumors that are resistant to 5-FU or other various drugs.

### BEST MODE FOR CARRYING OUT THE INVENTION

Examples are given below to illustrate the invention in more detail, but the scope of the invention is not limited by the Examples.

### Pharmacological Test Example 1

### Formulation of Pharmaceutical Preparations

A tegafur/gimeracil/oteracil potassium compound pharmaceutical agent (tegafur/gimeracil/oteracil potassium molar ratio=1:0.4:1, hereinafter sometimes referred to as TS-1), a tegafur/uracil compound pharmaceutical agent (tegafur/uracil molar ratio=1:4, hereinafter sometimes referred to as UFT), and d,l-calcium folinate were independently dissolved or suspended in 0.5% hydroxypropylmethyl cellulose (HPMC) solutions, and pharmaceutical preparations were formulated so as to allow administration thereof in a final dosage of 10 mL/kg. For example, if tegafur was to be administered in an amount of 8.3 mg/kg, a pharmaceutical agent corresponding to 8.3 mg of tegafur was dissolved or suspended in 10 mL of HPMC, and an oral pharmaceutical preparation was then formulated such that the drug could be administered in an amount of 10 mL/kg.

l-OHP was dissolved in 5% aqueous glucose solution (Otsuka Glucose, manufactured by Otsuka Pharmaceutical Factory Inc.), thereby giving an l-OHP preparation.

### Administration of Pharmaceutical Preparations

A fragment measuring about 2 mm cubic fragment of the human colon cancer xenograft KM20C strain was subcutaneously implanted on the backs of male nude rats F344/NJcl-rnu. When the average tumor volume (=0.5×major axis (mm)×minor axis (mm)²) reached about 200 mm³, the rats were divided into groups (day 0).

The TS-1 preparation or the UFT+LV preparation prepared above was orally administered once a day in amounts presented in Table 1 from the day after grouping (day 1) for 14 consecutive days. The l-OHP preparation was administered into the tail vein on day 1 at 10 mg/kg immediately before the administration of the aforementioned preparations.

The ratio of the tumor volume upon grouping (day 0) to the tumor volume on day 15 was regarded as the relative tumor volume: Tumor volume on day 15/Tumor volume on day 0

The extent of tumor growth inhibition (%) was calculated using the average relative tumor volume of the drug-administered groups and the average relative tumor volume of the control group:

[1−(Relative tumor volume of drug-administered group)/(Relative tumor volume of tumor-bearing control group)]×100 (%)

Moreover, the effect due to the combined use was analyzed using the relative tumor volumes on day 15 according to the IUT procedure (Intersection-Union test) (see Statistical Science 1996, Vol. 11, No. 4, 283-319).

The difference in body weight between rats on day 0 and rats on day 15 presented as a ratio (extent of body weight change) was used as an index of systemic toxicity of the pharmaceutical preparation:

[(Body weight of rats on day 15−Body weight of rats on day 0)/body weight of rats on day 0]×100 (%)

The results thus obtained are presented on Table 1. Although calcium folinate was used in the experiment, the dosage given in the table is that calculated as the equivalent amount of folinic acid.

TABLE 1

| | Dosage of | | Relative | Extent of | Extent of |
|---|---|---|---|---|---|
| | compound agent | Dosage of | tumor | tumor growth | body |
| | containing tegafur | oxaliplatin | volume | inhibition | weight |
| Drug | (mg/kg/day) | (mg/kg/day) | (Mean) | (%) | change (%) |
| None | — | — | 5.81 | — | 1.3 |
| Oxaliplatin (l-OHP) | — | 10 | 4.64 | 20.1 | 0.9 |
| Tegafur + gimeracil + | 12 + 3.5 + 11.7 | — | 2.83 | 51.4 | −3.4 |
| oteracil potassium | | | | | |
| (TS-1) | | | | | |
| Tegafur + gimeracil + | 12 + 3.5 + 11.7 | 10 | 2.13 *^{, ##} | 63.3 | −6.4 |
| oteracil potassium + | | | | | |
| oxaliplatin | | | | | |
| (TS-1 + l-OHP) | | | | | |
| Tegafur + uracil + | 24 + 53.8 + 20.0 | — | 3.73 | 35.8 | −3.1 |
| folinic acid | | | | | |
| (UFT + LV) | | | | | |
| Tegafur + uracil + | 24 + 53.8 + 20.0 | 10 | 2.79 * | 51.9 | −4.5 |
| folinic acid + | | | | | |
| oxaliplatin | | | | | |
| (UFT + LV + l-OHP) | | | | | |

* Group with a significant effect determined according to the IUT procedure (p < 0.05)
^{##} Group with a significant effect compared to a group given UFT + LV in combination with the same amount of l-OHP (p < 0.01)

The results presented above establish that the administration of TS-1 in combination with l-OHP is an extremely effective therapeutic method since it provides statistically significant effects when compared with the administration of TS-1 alone, and, when compared with the administration of UFT+LV in combination with l-OHP, affords statistically superior effects without substantially increasing toxicity.

### Pharmacological Test Example 2

### Action Toward a 5-FU-Resistant Strain

A fragment measuring about 2 mm cubic fragment of a 5-FU-resistant strain of the human colon cancer xenograft KM12C strain established by the applicant was subcutaneously implanted on the backs of male nude mice BALB/c-nu/nu. When the average tumor volume (=0.5×major axis (mm)×minor axis (mm)²) reached about 200 mm³, the mice were divided into groups (day 0). The TS-1 preparation formulated in Pharmacological Test Example 1 was orally administered to the mice once a day in an amount as shown in Table 2 from day 1 for 9 consecutive days. The l-OHP preparation was administered into the tail vein on day 1 at 8.3 mg/kg immediately before the administration of the TS-1 preparation. The ratio of the tumor volume on day 0 to the tumor volume on day 10 was calculated to obtain a relative tumor volume. As in Pharmacological Test Example 1, the extent of tumor growth inhibition and the extent of body weight change were calculated using the average relative tumor volumes of the drug-administered groups and the control group.

TABLE 2

| | Dosage of | Dosage of | Relative | | Extent of |
|---|---|---|---|---|---|
| | tegafur- | oxaliplatin- | tumor | Extent of | body |
| | containing agent | containing agent | volume | tumor growth | weight |
| Drug | (mg/kg/day) | (mg/kg/day) | (Mean) | inhibition (%) | change (%) |
| None | — | — | 9.5 | — | −6.2 |
| Oxaliplatin | — | 8.3 | 7.97 | 16.1 | −8.0 |
| (l-OHP) | | | | | |
| Tegafur + | 8.3 + 2.4 + 8.1 | — | 6.39 | 32.7 | −9.2 |
| gimeracil + | | | | | |
| oteracil potassium | | | | | |
| (TS-1) | | | | | |
| Tegafur + | 8.3 + 2.4 + 8.1 | 8.3 | 4.89 * | 48.5 | −18.6 |
| gimeracil + | | | | | |
| oteracil potassium + | | | | | |
| oxaliplatin | | | | | |
| (TS-1 + l-OHP) | | | | | |

* Group with a significant effect determined according to the IUT procedure (p < 0.05)

The results presented above establish that the administration of TS-1 in combination with l-OHP is an effective therapeutic method toward 5-FU-resistant strains since, while TS-1 alone barely exhibits any effect on the 5-FU-resistant strain, the administration of TS-1 in combination with l-OHP potentiated the extent of tumor growth inhibition to essentially the same extent (about 50%) as with the 5-FU-sensitive strains, without accompanied by a significant body weight change.

### Pharmacological Test Example 3

### Action Toward a Multidrug-Resistant Strain

A fragment measuring about 2 mm cubic fragment of the human colon cancer xenograft HCT-15 strain, which expresses P-glycoprotein in large amounts and is resistant to multiple drugs, was subcutaneously implanted on the backs of male nude mice BALB/c-nu/nu. When the average tumor volume (=0.5×major axis (mm)×minor axis (mm)²) reached about 200 mm³, the mice were divided into groups (day 0). A TS-1 preparation as prepared in Pharmacological Test Example 1 was orally administered to the mice once a day in an amount as shown in Table 3 from day 1 for 14 consecutive days. The l-OHP preparation was administered only once into the tail vein on day 1 at 10.0 mg/kg immediately before the administration of the TS-1 preparation. The extent of tumor growth inhibition was calculated as in Pharmacological Test Example 1. Using the relative tumor volumes on day 15, the difference in relative tumor volume between the control group and the drug-administered groups was determined according to a Dunnett's test, and the difference between the one which was given either TS-1 or l-OHP and the one which was given TS-1 and l-OHP in combination was evaluated according to Student's t-test.

TABLE 3

| | Dosage of compound | | Relative | |
|---|---|---|---|---|
| | agent containing | Dosage of | tumor | Extent of tumor |
| | tegafur | oxaliplatin | volume | growth |
| Drug | (mg/kg/day) | (mg/kg/day) | (Mean) | inhibition (%) |
| None | — | — | 4.53 | — |
| Oxaliplatin | — | 10 | 4.33 | 4.3 |
| (l-OHP) | | | | |
| Tegafur + gimeracil + | 10 + 2.9 + 9.8 | — | 2.56 * | 43.4 |
| oteracil potassium | | | | |
| (TS-1) | | | | |
| Tegafur + gimeracil + | 10 + 2.9 + 9.8 | 10 | 1.54 **^{, #, $} | 66.0 |
| oteracil potassium + | | | | |
| oxaliplatin | | | | |
| (TS-1 + l-OHP) | | | | |

* Group with a significant effect relative to the tumor-bearing control group (p < 0.05)
** Group with a significant effect relative to the tumor-bearing control group (p < 0.01)
^{#} Group with a significant effect relative to the one which was given TS-1 alone (p < 0.025)
^{$} Group with a significant effect relative to the one which was given oxaliplatin alone (p < 0.01)

As demonstrated above, the administration of TS-1 in combination with l-OHP significantly potentiated the antitumor effect of TS-1 on a tumor on which l-OHP alone barely shows any antitumor effect. The results therefore establish that the potentiation of antitumor effect is due to the antitumor effect potentiating activity of l-OHP toward TS-1. The results suggest that a combination therapy using TS-1 and l-OHP is effective against tumors that are insensitive to a large number of anticancer preparations since the tumor used herein is a multidrug-resistant tumor.

### Pharmacological Test Example 4

### Dose Dependency of l-OHP

A fragment measuring about 2 mm cubic fragment of the human colon cancer xenograft COL-1 strain was subcutaneously implanted on the backs of male nude mice BALB/c-nu/nu. When the average tumor volume (=0.5×major axis (mm)×minor axis (mm)²) reached about 125 mm³, the mice were divided into groups (day 0). The TS-1 preparation formulated in Pharmacological Test Example 1 was orally administered to the mice once a day in an amount equivalent to 6.9 mg/kg of tegafur from the day after grouping for 14 consecutive days. l-OHP dissolved in 5% aqueous glucose solution was administered into the tail vein on days 1 and 8 at 2.8, 3.5, 4.2, or 5.0 mg/kg/day immediately before the administration of the TS-1 preparation.

For the determination of antitumor effect, the ratio of the tumor volume upon grouping (day 0) to the tumor volume on day 15 was calculated to obtain a relative tumor volume. The extent of tumor growth inhibition was determined using the average relative tumor volumes of the drug-administered groups and the control group. Using the relative tumor volumes on the evaluation day, the statistical significance of the difference in relative tumor volume between the control group and the one which was given the TS-1 preparation alone was analyzed according to Student's t-test; the correlation between the antitumor effect by l-OHP and the dosage thereof was analyzed according to a Williams' test; the statistical significance of the difference in relative tumor volume between the control group and the one which was given l-OHP alone was analyzed according to a Dunnett's test; and the effect observed in the one which was given TS-1 and l-OHP in combination relative to the one which was given either TS-1 or l-OHP was analyzed according to the IUT procedure.

The extent of body weight change between the mice on day 0 and the mice on day 15 was used as an index of systemic toxicity of l-OHP.

TABLE 4

| | Dosage of compound | Dosage of | Relative tumor | Extent of | Extent of | Number of |
|---|---|---|---|---|---|---|
| | agent containing | oxaliplatin | volume | tumor growth | body weight | dead |
| Drug | tegafur (mg/kg) | (mg/kg) | (Mean) | inhibition (%) | change (%) | animal |
| None | — | — | 5.75 | — | −16.1 | — |
| Tegafur + gimeracil + | 6.9 + 2.0 + 6.8 | — | 3.80 ** | 33.9 | −20.5 | 0 |
| oteracil potassium (TS-1) | | | | | | |
| Oxaliplatin (l-OHP) | — | 2.9 | 5.06 ^{§} | 11.9 | −18.0 | 0 |
| l-OHP | — | 3.5 | 4.54 ^{§,} * | 21.0 | −20.6 | 0 |
| l-OHP | — | 4.2 | 4.43 ^{§,} ** | 23.0 | −19.0 | 0 |
| l-OHP | — | 5.0 | 4.41 ^{§,} ** | 23.3 | −16.8 | 0 |
| TS-1 + l-OHP | 6.9 + 2.0 + 6.8 | 2.9 | 3.70 ^{#} | 35.7 | −20.1 | 0 |
| TS-1 + l-OHP | 6.9 + 2.0 + 6.8 | 3.5 | 2.55 ^{#} | 55.6 | −19.3 | 0 |
| TS-1 + l-OHP | 6.9 + 2.0 + 6.8 | 4.2 | 2.65 ^{#} | 53.9 | −22.0 | 0 |
| TS-1 + l-OHP | 6.9 + 2.0 + 6.8 | 5.0 | 2.34 ^{#} | 59.3 | −23.4 | 1 |

* Group with a significant effect relative to the control group (p < 0.05)
** Group with a significant effect relative to the control group (p < 0.01)
^{§} Group with an effect significantly correlated with dosage (p < 0.05)
^{#} Group with a significant effect by a combined use determined according to the IUT procedure (p < 0.05)

The results given above establish that the effect of the tegafur/gimeracil/oteracil potassium compound pharmaceutical agent is proportional to the dosage of l-OHP.

### Pharmacological Test Example 5

### Test for Efficacy Comparison

### Preliminary Test (Investigation of Maximum Tolerable Dose of Capecitabine)

A fragment measuring about 2 mm cubic fragment of the human colon cancer xenograft COL-1 strain was subcutaneously implanted on the backs of male nude mice BALB/c-nu/nu. When the average tumor volume (=0.5×major axis (mm)×minor axis (mm)²) reached about 190 mm³, the mice were divided into groups (day 0). Capecitabine suspended in 0.5% HPMC solution was orally administered to the mice once a day at 240, 360, or 540 mg/kg from the day after grouping for 14 consecutive days. l-OHP dissolved in 5% aqueous glucose solution was administered into the tail vein on days 1 and 8 at 4.2 mg/kg/day immediately before the administration of the Capecitabine. The difference in body weight between the mice on day 0 and the mice on day 15 presented as a ratio and the occurrence of death were used as indices of systemic toxicity of capecitabine. The results showed that the maximum tolerable dose at which death due to toxicity is not observed of capecitabine is 360 mg/kg/day.

### Main Test (Test for Efficacy Comparison)

A fragment measuring about 2 mm cubic fragment of the human colon cancer xenograft COL-1 strain was subcutaneously implanted on the backs of male nude mice BALB/c-nu/nu. When the average tumor volume (=0.5×major axis (mm)×minor axis (mm)²) reached about 170 mm³, the mice were divided into groups (day 0). The TS-1 preparation formulated in Pharmacological Test Example 1 was orally administered to a group of mice once a day from the day after grouping for 14 consecutive days in an amount equivalent to the maximum tolerable dose of tegafur, i.e., 6.9 mg/kg. l-OHP dissolved in 5% aqueous glucose solution was administered into the tail vein on days 1 and 8 in the maximum tolerable dose, i.e., 4.2 mg/kg/day immediately before the administration of the TS-1 preparation. Capecitabine suspended in 0.5% HPMC solution was orally administered to another group of mice once a day from the day after grouping for 14 consecutive days in the maximum tolerable amount determined in the preliminary test, i.e., 360 mg/kg. l-OHP dissolved in 5% aqueous glucose solution was administered into the tail vein on days 1 and 8 at 4.2 mg/kg/day immediately before the administration of the Capecitabine.

For the determination of antitumor effect, the ratio of the tumor volume upon grouping (day 0) to the tumor volume on day 15 was calculated to obtain a relative tumor volume. The extent of tumor growth inhibition was determined using the average relative tumor volumes of the drug-administered groups and the control group. Using the relative tumor volumes on the test day, the statistical significance of the difference in relative tumor volume between the control group and the one which was given the pharmaceutical agents was analyzed according to a Dunnett's test; and the statistical significance of the difference in relative tumor volume between the one which was given TS-1 and l-OHP in combination and the one which was given capecitabine and l-OHP in combination was analyzed according to Student's t-test. The extent of body weight change between the mice on day 0 and the mice on day 15 was used as an index of systemic toxicity of the pharmaceutical preparations.

TABLE 5

| | Dosage of | | | | | |
|---|---|---|---|---|---|---|
| | fluorinated | Dosage of | Time of oxaliplatin | Relative tumor | Extent of tumor | Extent of body |
| | pyrimidine | oxaliplatin | administration | volume | growth inhibition | weight change |
| Drug | (mg/kg) | (mg/kg) | (day) | (Mean) | (%) | (%) |
| None | — | — | — | 5.42 | — | −14.8 |
| Tegafur + gimeracil + oteracil | 6.9 + 2.0 + 6.8 | 4.2 | 1, 8 | 1.87 ***^{, ###} | 65.5 | −24.0 |
| potassium + oxaliplatin | | | | | | |
| (TS-1 + l-OHP) | | | | | | |
| Capecitabine + oxaliplatin | 360 | 4.2 | 1, 8 | 2.94 *** | 45.7 | −26.4 |

*** Group with a significant effect relative to the control group (p < 0.001)
^{###} Group with a significant effect relative to the one which was given capecitabine + oxaliplatin (p < 0.001)

The test for efficacy comparison at maximum tolerable doses described above demonstrated that a combination therapy using a tegafur/gimeracil/oteracil potassium compound pharmaceutical agent and l-OHP shows a clearly better antitumor effect than a combination therapy using capecitabine and l-OHP. The systemic toxicities (body weight losses) were almost identical.

The results of the Test Examples presented above reveal that a combination therapy using a tegafur/gimeracil/oteracil potassium compound pharmaceutical agent and l-OHP significantly potentiates antitumor activity without substantially aggravating side effects compared with the use of a tegafur/gimeracil/oteracil potassium compound pharmaceutical agent alone. The results also demonstrate that, in terms of antitumor effect, the combination therapy is a significantly more effective therapeutic method compared with a standard therapy in which a tegafur/uracil compound pharmaceutical agent and d,l-calcium folinate as well as capecitabine are used in combination. Although it has been considered that, in the case of cancers recurring after therapies mainly using fluorinated pyrimidines, repeating such therapies in which fluorinated pyrimidines are mainly used are likely to be fruitless, the results of Pharmacological Test Example 2 establish that a tegafur/gimeracil/oteracil potassium compound pharmaceutical agent can be advantageously and continuously used in such treatments without substantially aggravating side effects if used in combination with l-OHP. Furthermore, it is herein indicated that potentiation in antitumor effect attained when l-OHP is used in combination is due to the antitumor effect potentiating action of l-OHP toward the tegafur/gimeracil/oteracil potassium compound pharmaceutical agent since a significant antitumor effect was obtained when a tegafur/gimeracil/oteracil potassium compound pharmaceutical agent was used in combination with l-OHP, even on a tumor for which l-OHP by itself does not show any antitumor effect. The results of the test using a multidrug-resistant tumor establish that a combination therapy with a tegafur/gimeracil/oteracil potassium compound pharmaceutical agent and l-OHP is effective toward tumors that are not sensitive (i.e., that are resistant) to a large number of anticancer agents.

### FORMULATION EXAMPLES

Formulation examples of the antitumor effect potentiator and the antitumor preparation of the present invention are given below.

### Formulation Example 1

### Injectable Solution

| | | |
|---|---|---|
| l-OHP | 100 | mg |
| 5% Aqueous glucose solution | 50 | mL |
| Amount per ampule | 50 | mL |

Injectable solutions were prepared according to a standard method using the formulation presented above.

### Formulation Example 2

### Granule

| | | |
|---|---|---|
| Tegafur | 50 | mg |
| Gimeracil | 14.5 | mg |
| Oteracil potassium | 49 | mg |
| l-OHP | 55 | mg |
| Lactose | 280 | mg |
| Cornstarch | 298 | mg |
| Hydroxypropylmethyl cellulose | 10 | mg |
| Amount per wrapper | 756.5 | mg |

Granules were prepared according to a standard method using the formulation presented above.

### Formulation Example 3

### Capsule

| | | |
|---|---|---|
| Tegafur | 25 | mg |
| Gimeracil | 7.25 | mg |
| Oteracil potassium | 24.5 | mg |
| l-OHP | 40 | mg |
| Lactose | 51 | mg |
| Crystalline cellulose | 28 | mg |
| Magnesium stearate | 5 | mg |
| Amount per capsule | 180.75 | mg |

Capsules were prepared according to a standard method using the formulation presented above.

### Formulation Example 4

### Tablet

| | | |
|---|---|---|
| Tegafur | 20 | mg |
| Gimeracil | 5.8 | mg |
| Oteracil potassium | 19.6 | mg |
| l-OHP | 39.6 | mg |
| Lactose | 51 | mg |
| Crystalline cellulose | 15 | mg |
| Magnesium stearate | 3 | mg |
| Cornstarch | 14 | mg |
| Hydroxypropylmethyl cellulose | 10 | mg |
| Amount per tablet | 178.0 | mg |

Tablets were prepared according to a standard method using the formulation presented above.

### Formulation Example 5

### Suppository

| | | |
|---|---|---|
| Tegafur | 200 | mg |
| Gimeracil | 58 | mg |
| Oteracil potassium | 196 | mg |
| l-OHP | 396 | mg |
| Witepsol W-35 | 1150 | mg |
| Amount per suppository | 2000 | mg |

Suppositories were prepared according to a standard method using the formulation presented above.

1. An antitumor effect potentiator for potentiating antitumor activity of an antitumor preparation comprising tegafur in a therapeutically effective amount, gimeracil in an amount effective for potentiating an antitumor effect, and oteracil potassium in an amount effective for inhibiting a side effect, the antitumor effect potentiator comprising cis-oxalate(1R,2R-diaminocyclohexane)platinum(II) in an amount effective for potentiating an antitumor effect.

2. The antitumor effect potentiator according to claim 1, wherein the antitumor preparation comprises tegafur, gimeracil, and oteracil potassium in a molar ratio of 1:0.4:1.

3. A method for treating cancer comprising the step of administering to a mammal the combination of tegafur in a therapeutically effective amount, gimeracil in an amount effective for potentiating an antitumor effect, oteracil potassium in an amount effective for inhibiting a side effect, and cis-oxalate(1R,2R-diaminocyclohexane)platinum(II) in an amount effective for potentiating an antitumor effect.

4. The method for treating cancer according to claim 3, wherein, per mole of tegafur, gimeracil is used in a proportion of 0.1 to 5 mole, oteracil potassium is used in a proportion of 0.1 to 5 mole, and cis-oxalate(1R,2R-diaminocyclohexane)platinum(II) is used in a proportion of 0.1 to 5 mole.

5. The method for treating cancer according to claim 4, wherein tegafur, gimeracil, oteracil potassium, and cis-oxalate(1R,2R-diaminocyclohexane)platinum(II) are used in a molar ratio of 1:0.4:1:0.1 to 5.

6. An antitumor preparation in a pharmaceutical form comprising a plurality of pharmaceutical agents each of which contains one active ingredient selected from the group consisting of tegafur, gimeracil, oteracil potassium, and cis-oxalate(1R,2R-diaminocyclohexane)platinum(II), or each of which contains such active ingredients in any combination, or in a pharmaceutical form comprising a single pharmaceutical agent containing all of the active ingredients.

7. The antitumor preparation according to claim 6 in a pharmaceutical form comprising a compound pharmaceutical agent comprising 3 ingredients, i.e., tegafur, gimeracil, and oteracil potassium, as active ingredients, and a pharmaceutical agent comprising cis-oxalate(1R,2R-diaminocyclohexane)platinum(II) as an active ingredient.

8. The antitumor preparation according to claim 6, wherein the proportion of the active ingredients is such that, per mole of tegafur, gimeracil is used in a proportion of 0.1 to 5 mole, oteracil potassium is used in a proportion of 0.1 to 5 mole, and cis-oxalate(1R,2R-diaminocyclohexane)platinum(II) is used in a proportion of 0.1 to 5 mole.

9. The antitumor preparation according to claim 8, wherein the molar ratios of the active ingredients are such that tegafur, gimeracil, oteracil potassium, and cis-oxalate(1R,2R-diaminocyclohexane)platinum(II) are used in a molar ratio of 1:0.4:1:0.1 to 5.

10. A kit comprising a combination of pharmaceutical compositions for treating cancer in a mammal comprising (a) an antitumor composition comprising tegafur in a therapeutically effective amount, gimeracil in an amount effective for potentiating an antitumor effect, and oteracil potassium in an amount effective for inhibiting a side effect, and (b) cis-oxalate(1R,2R-diaminocyclohexane)platinum(II) in an amount effective for potentiating an antitumor effect.

11. A method for potentiating an antitumor effect of an antitumor preparation, the method comprising the step of administering cis-oxalate(1R,2R-diaminocyclohexane)platinum(II) in an amount effective for potentiating an antitumor effect in combination with an antitumor preparation comprising tegafur in a therapeutically effective amount, gimeracil in an amount effective for potentiating an antitumor effect, and oteracil potassium in an amount effective for inhibiting a side effect.

12. The method according to claim 11, wherein cis-oxalate(1R,2R-diaminocyclohexane)platinum(II) is administered concurrently with, or within 4 hours before or after, the administration of the antitumor preparation comprising tegafur in a therapeutically effective amount, gimeracil in an amount effective for potentiating an antitumor effect, and oteracil potassium in an amount effective for inhibiting a side effect.

13. Use of cis-oxalate(1R,2R-diaminocyclohexane)platinum(II) in production of an antitumor effect potentiator to potentiate an antitumor effect of an antitumor preparation comprising tegafur in a therapeutically effective amount, gimeracil in an amount effective for potentiating an antitumor effect, and oteracil potassium in an amount effective for inhibiting a side effect.

14. Use of cis-oxalate(1R,2R-diaminocyclohexane)platinum(II) in production of an antitumor preparation comprising tegafur in a therapeutically effective amount, gimeracil in an amount effective for potentiating an antitumor effect, and oteracil potassium in an amount effective for inhibiting a side effect, to produce an antitumor preparation that has an potentiated antitumor effect.
